(19) United States
(12) Patent Application Publication
(10) Pub. No.: US 2025/0166802 A1
(43) Pub. Date: May 22, 2025

(54) SYSTEM AND METHOD TO MATCH PARTIAL PATIENT DATA FROM DIFFERENT SOURCES TO CREATE A COMPLETE PICTURE OF PATIENT CARE

(71) Applicant: KONINKLIJKE PHILIPS N.V., EINDHOVEN (NL)

(72) Inventors: EKIN KOKER, CAMBRIDGE, MA (US); SIVA CHAITANYA CHADUVULA, MALDEN, MA (US); RANJITH NAVEEN TELLIS, TEWKSBURY, MA (US); SANDEEP MADHUKAR DALAL, WINCHESTER, MA (US); OLGA STAROBINETS, NEWTON, MA (US); SAIFENG LIU, CAMBRIDGE, MA (US); THOMAS ERIK AMTHOR, HAMBURG (DE); FALK UHLEMANN, NORDERSTEDT (DE)

(21) Appl. No.: 18/841,716

(22) PCT Filed: Feb. 22, 2023
(86) PCT No.: PCT/EP2023/054431

§ 371 Date: Aug. 27, 2024

Related U.S. Application Data
(60) Provisional application No. 63/315,223, filed on Mar. 1, 2022.

(51) Int. Cl. G16H 40/20; A61B 5/00; G16H 10/60; G16H 30/20

(52) U.S. Cl. CPC G16H 40/20; A61B 5/0077; G16H 10/60; G16H 30/20

(57) ABSTRACT

Systems, apparatuses, and methods provide for matching unassigned patient data to individual patients. For example, such operations include collecting data from a plurality of data sources in a plurality of formats. Data information Machine time stamps are converted from collected data to universal time zone data time stamps. A same patient is matched to the collected data based on the universal time zone data time stamp. A quality estimate of the match is quantified. The match and quality estimate of the match are transferred to a user interface.

### FIELD

The following relates generally to medical records. More particularly, embodiments herein relate to matching unassigned patient data to individual patients.

### BACKGROUND

In patient care environments there is often patient data coming from various sources. Often, such patient data is unassigned (e.g., the patient data coming from various sources has no exact match with a patient or has no patient identifying information). Machine logs and first and third party sensors (e.g., camera, radar, etc.) may have information about individual exams but no context about the patient.

Healthcare platforms (e.g., Performance Management Data Platform, Virtualized Imaging Solution, Interoperability Solution, Electronic Medical Record (EMR), radiology information systems (RIS), picture archiving and communication system (PACS), etc.) typically have information about patients but not about various activities patient goes through in an exam room. Accordingly, there is often inherent uncertainty around the validity of associating the data with an individual patient (e.g., a patient is scheduled to a different room than the original plan, a staff member called in sick, so another had to step in, etc.).

For example, in a radiology context, radiology information systems (RIS) have information about a patient, picture archiving and communication system (PACS) has all of the images, machine logs have the information about the imaging protocols and timestamps of various activities that happen while that patient was on the machine. But none of these systems connect the dots fully to create a complete record of the patient care. Accordingly, clinicians/care providers typically have to manually sift through this data to understand the patient's past and their current needs in a particular context.

In general, data for the same patient is often kept in different information silos and these separate bucket of data are not typically being matched. Current methods of matching the patient data from various sources is often done manually by sifting through logs, phone calls, conversations with different people in the hospital by nurses/clinicians/operations managers. Such operations are laborious, prone to human error, and humans may not have ready access to machine logs and the like.

The following discloses certain improvements to overcome these problems and others.

### SUMMARY

As discussed above, it is currently challenging to utilizing unassigned patient data as a reliable source patient care and diagnosis. Accordingly, there is a need for matching patient data coming from various sources when there is no exact patient match.

As will be described in greater detail below, in some implementations discussed herein, an automated tool may advantageously be used to address the challenges in utilizing unassigned patient data. Systems, apparatuses, and methods are described herein to provide for matching unassigned patient data to individual patients.

In some implementations discussed herein, procedures are described that connect partial operational and clinical data from various sources currently stored in separate systems/silos and match them to a patient record to provide a complete picture of patient care. For example, such procedures employ temporal matching algorithms, statistical and machine learning methods to clean up and standardize the data, cluster timestamps belonging to the same patient, etc. to provide a probabilistic estimate of the quality of a match between a specific patient and unassigned patient data. Collected data can be used to improve operational workflows in real-time by operational managers and/or can be viewed retrospectively by clinicians to understand the previous care steps a patient went through to make more informed decisions for patient's care.

In a patient care setting, there may be many platforms (e.g., Performance Management Data Platform, Medical Asset Track & Trace System, Virtualized Imaging Solution, Centralized Care Management System, Interoperability Solution, Electronic Medical Record (EMR), radiology information systems (RIS), picture archiving and communication system (PACS), etc.) connected to various clinical and operational data sources (e.g., Health Level Seven (HL7), Fast Healthcare Interoperability Resources (FHIR), machine logs, Real-Time Location System (RTLS), first and third party sensors, cameras, etc.). Currently, all of these data sources typically only have partial data about individual patients due to the limitations of the technology or platform and none have a complete timeline of care on a patient-by-patient basis.

In one example, an implementation aims at providing workflow forecasting, simulation, and optimization capabilities. In another example, an implementation aims at virtualizing imaging operations through connecting expert users (e.g., technologists, radiologists, etc.) at a hub site with the local technologists at the spoke sites remotely with an Integrated Delivery Network (IDN) for over-the-shoulder support and remote guidance.

In one aspect, an apparatus includes a data collector, a data cleaner, a temporal match module, and a match quantifier. The data collector collects data from a plurality of data sources in a plurality of formats. The data cleaner is communicatively coupled to the data collector to convert data time stamps from the collected data to universal time zone data time stamps. The temporal match module is communicatively coupled to the data cleaner to match a same patient to the collected data based on the universal time zone data time stamps. The match quantifier is communicatively coupled to the temporal match module and a user interface. The match quantifier is to quantify a quality estimate of the match, and to transfer the match and quality estimate of the match to the user interface.

In another aspect, a method includes collecting, via a data collector, data from a plurality of data sources in a plurality of formats. Data time stamps are converted, via a data cleaner communicatively coupled to the data collector, from collected data to universal time zone data time stamps. A same patient is matched, via a temporal match module communicatively coupled to the data cleaner, to the collected data based on the universal time zone data time stamps. A quality estimate of the match is quantified, via a match quantifier communicatively coupled to the temporal match module and a user interface. The match and quality estimate of the match are transferred, via the match quantifier, to the user interface.

In yet another aspect, a machine-readable storage includes machine-readable instructions, which when executed, include operations to collect data from a plurality of data sources in a plurality of formats. Data time stamps are converted from collected data to universal time zone data time stamps. A same patient is matched to the collected data based on the universal time zone data time stamp. A quality estimate of the match is quantified. The match and quality estimate of the match are transferred to a user interface.

In still another aspect, an apparatus includes means for collecting data from a plurality of data sources in a plurality of formats. The apparatus also includes means for converting data time stamps from collected data to universal time zone data time stamps. The apparatus also includes means for matching a same patient to the collected data based on the universal time zone data time stamp. The apparatus also includes means for quantifying a quality estimate of the match. The apparatus also includes means for transferring the match and quality estimate of the match are transferred to a user interface.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

The various advantages of the embodiments will become apparent to one skilled in the art by reading the following specification and appended claims, and by referencing the following drawings, in which:

FIG. 1 is an illustration of a block diagram of an example unassigned patient data management system according to an embodiment;

FIG. 2 is an illustration of a block diagram of another example unassigned patient data management system according to an embodiment;

FIG. 3 is an illustration of a flowchart of a method for matching unassigned patient data to individual patients according to an embodiment;

FIGS. 4A and 4B is an illustration of a flowchart of a further method for matching unassigned patient data to individual patients according to an embodiment;

FIG. 5 is an illustration of a block diagram of a computer program product according to an embodiment;

FIG. 6 is a further illustration of a EMR management system according to an embodiment; and

FIG. 7 is an illustration of a hardware apparatus including a semiconductor package according to an embodiment.

### DETAILED DESCRIPTION

Timestamps are typically the most common element across various data sources. Some implementations described herein are directed to the use of temporal matching algorithms to connect various pieces of partial information about the patient that comes from different sources to create a complete picture of the patient care.

As will be described in greater detail below, in some implementations discussed herein, an automated tool may advantageously be used to address the challenges in utilizing unassigned patient data. Systems, apparatuses, and methods are described herein to provide for matching unassigned patient data to individual patients. For example, such operations include collecting data from a plurality of data sources in a plurality of formats. Data time stamps are converted from collected data to universal time zone data time stamps. A same patient is matched to the collected data based on the universal time zone data time stamp. A quality estimate of the match is quantified. The match and quality estimate of the match are transferred to a user interface.

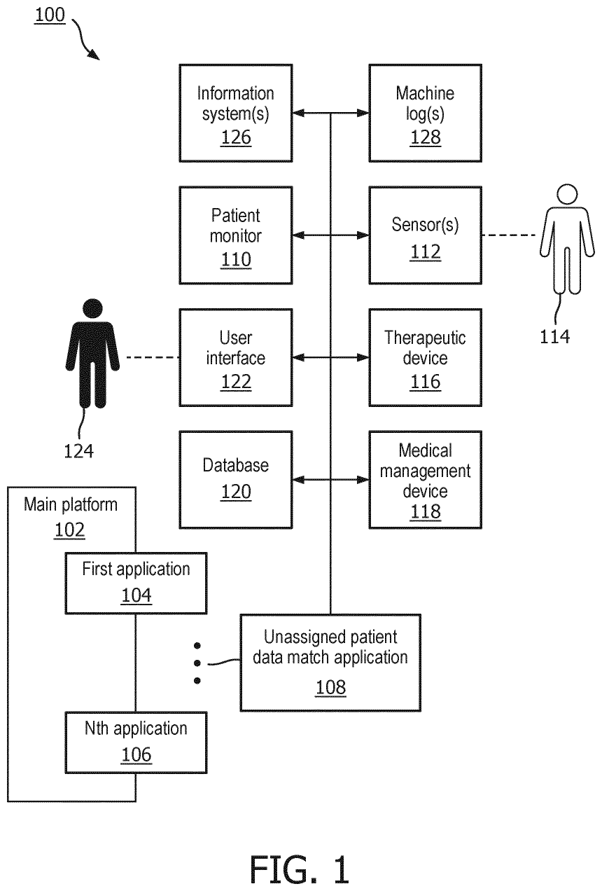

FIG. 1 is an illustration of a block diagram of an example unassigned patient data management system 100 according to an embodiment. For example, the unassigned patient data management system 100 may be centralized or may be distributed and may include some or all elements and components of one or more computers or computer systems.

In the illustrated implementation, the unassigned patient data management system 100 may include a main platform 102 (e.g., also referred to as a care platform herein). In some implementations, the main platform 102 may be embodied as a server computer or a plurality of server computers (e.g., interconnected to form a server cluster, cloud computing resource, the like, and/or combinations thereof).

In some implementations, the main platform 102 is a care platform designed for one or more aspects of patient care. In such an example, the main platform 102 includes one or more of the following care platforms Performance Management Data Platform, Medical Asset Track & Trace System, Virtualized Imaging Solution, Centralized Care Management System, Interoperability Solution, Electronic Medical Record (EMR), radiology information systems (RIS), picture archiving and communication system (PACS), etc. In a patient care setting, there may be many care platforms connected to various clinical and operational data sources (e.g., Health Level Seven (HL7), Fast Healthcare Interoperability Resources (FHIR), machine logs, Real-Time Location System (RTLS), first and third party sensors (e.g., including cameras), etc.). As described above, all of these data sources typically only have partial data about individual patients due to the limitations of the technology or platform and none have a complete timeline of care on a patient-by-patient basis.

In some implementations, a first application 104 though an Nth application 106, which may include an unassigned patient data match application 108, may be associated with the main platform 102. The operations of the unassigned patient data match application 108 trigger unassigned patient data notifications to a clinician in response to a match being made of patient data to a particular patient. Additionally, or alternatively, the unassigned patient data match application 108 may trigger notifications of clinical interventions in response to a match being made of patient data to a particular patient.

Additionally, or alternatively, the unassigned patient data management system 100 may include a patient monitor 110, a sensor 112 (e.g., one or more sensors 112 that may be associated with a care facility, sensors 112 that may be associated with a patient 114, the like, and/or combinations thereof), a therapeutic device 116, a medical management device 118, a database 120, a user interface 122 (e.g., one or more user interfaces 122 may be associated with a user 124), an information system 126, a machine log 128, the like, and/or combinations thereof. For example, the main platform 102, the patient monitor 110, the sensor 112, the therapeutic device 116, the medical management device 118, the database 120, the user interface 122, the information system 126, and/or the machine log 128 may be in communication with one another via Internet based communicating, cloud based communication, wired communication, wireless communication, the like, and/or combinations thereof.

In an example, the patient monitor 110 may be utilized to access patient data from and/or enter patient data to the database 120. For example, the patient monitor 110 may determine measured patient data (e.g., via one of more of the sensors 112). In such an example, the patient monitor 110 may be configured to monitor a patient for vital signs and the like, and the patient monitor 110 may communicate such measured patient data to the database 120. For example, the sensors 112 may determine dynamic patient condition data including patient vitals (e.g., blood pressure, pulse, temperature, respiration, and/or the like) and/or patient test results (e.g., creatine level, urine flow, potassium level, oxygen saturation, blood glucose level, carbon dioxide level, and/or the like).

In some implementations, the patient monitor 110 may comprise a bedside-type monitor, a transport-type monitor, a central station-type monitor, the like, and/or combinations thereof.

In some implementations, the sensors 112 may be minimally invasive-style sensors (e.g., by puncturing the skin, sensing through the skin, the like, and/or combinations thereof). Sensors 112 may be wired or wireless.

In some implementations, the sensors 112 may be associated with a care facility (e.g., as a facility monitor system). In such an example, the sensors 112 of a facility monitor system include first and/or third party cameras, video recorders, audio recorders, the like, and/or combinations thereof. For example, the facility monitor system includes de-identified human presence data. Such de-identified human presence data includes one or more of the following: a video-type camera feed, an infrared-type camera feed, a still-image type camera feed, an audio feed, the like, and/or combinations thereof. Additionally, or alternatively, the facility monitor system includes a Real-Time Location System (RTLS). For example, such a RTLS is a system used to provide immediate or real-time tracking and management of medical equipment, staff, and patients within a patient care environment via locating various tags, badges, the like, and/or combinations thereof. Such tags and/or badges may utilize one or more technology platforms (e.g., radio frequency (RF) communication, optical technology (e.g., infrared), acoustic (e.g., ultrasound) technology, etc.).

In some implementations, the sensors 112 may be associated with medical imaging. In such an example, the sensors 112 for medical imaging include one or more medical imaging devices, medical imaging systems, the like, and/or combinations thereof. For example, the sensors 112 for medical imaging include a magnetic resonance imaging (MRI) device, an ultrasound device, an x-ray device, a computerized tomography (CT) device, a radiology information systems (RIS), a patient, picture archiving and communication system (PACS), the like, and/or combinations thereof. In such an implementation, the sensors 112 are associated with standardized format medical imaging information to transmit, store, retrieve, print, process, and display medical imaging information (e.g., Digital Imaging and Communications in Medicine (DICOM) data).

In another example, the therapeutic device 116 may be utilized to access patient data from and/or enter patient data to the database 120. For example, the therapeutic device 116 may determine measured patient data. In such an example, the therapeutic device 116 may be configured to monitor the delivery of a particular therapy (e.g., a non-medication treatment) to a patient and may communicate such measured patient data to database 120.

In some implementations, the therapeutic device 116 may supply and/or monitor the administration of one or more patient procedures (e.g., dialysis).

In a further example, the medical management device 118 may be utilized to access patient data from and/or enter patient data to the database 120. For example, the medical management device 118 may determine measured patient data. In such an example, the medical management device 118 may be configured to monitor medication delivery to a patient and may communicate such measured patient data to the database 120.

In some implementations, the medical management device 118 may supply and/or monitor the administration of one or more patient medications (e.g., blood pressure medications, diuretic medications, anti-anemia medications, cholesterol lowering medications, vitamin supplements, and/or the like).

Additionally, or alternatively, in a still further example, the user interface 122 may be utilized to access patient data from and/or enter patient data to the database 120. In some implementations, user interface 122 may be implemented via one or more formfactor devices (e.g., a smart phone, a tablet, a laptop, a workstation, and/or the like), an interface associated with the main platform, and/or an interface associated with the patient monitor 110. Additionally, or alternatively, a care provider (e.g., user 124) may access patient data and/or enter patient data through an analog device, a non-networked patient monitor, a non-networked therapeutic device, a non-networked medical management device, the like, and/or combinations thereof.

In the illustrated implementation, the database 120 may include one or more types of patient data. For example, the database 120 may include patient data including medical images, laboratory result data, microbiology data, medication data, vital sign data, care order data, admission discharge and transfer data, and/or the like. As used herein, the term “database” refers to a collection of data and information organized in such a way as to allow the data and information to be stored, retrieved, updated, and/or manipulated. The term “database” as used herein may also refer to databases that may reside locally or that may be accessed from a remote location (e.g., via remote network servers).

As used herein, the term “patient data” refers to clinical data, imaging, or information related to an individual patient. Patient data may include measured patient data from a medical imaging device, an analog medical device, a sensor, a patient monitor, a therapeutic device, a medical management device, a medical imaging device, the like, and/or combinations thereof.

In the illustrated implementation, the information system 126 may include one or more types of patient data that are the same or in addition to the patient data of the database 120. For example, the information system 126 may be a Hospital Information System (HIS). Such a Hospital Information System (HIS) has patient data including Health Level Seven (HL7) data, Fast Healthcare Interoperability Resources (FHIR) data, the like, and/or combinations thereof.

In the illustrated implementation, the machine log 128 may include one or more types of patient data that are the same or in addition to the patient data of the database 120. For example, the machine log 128 may include patient data including logs from sensor 112 (e.g., medical imaging device machine logs, etc.).

Additionally, or alternatively, in some implementations, the database 120 may include or be associated with a simulated database. In such an example, such a simulated database may generate estimated patient data. For example, the simulated database may utilize some measured patient data from the patient monitor 110, the sensor 112, the therapeutic device 116, the medical management device 118, and/or the user interface 122 to generate some other estimated patient data. Such a simulated database may utilize digital twin technology to perform the estimation, for example. In such an example, such estimated patient data may be marked to indicate its estimated nature (rather than measured patient data). Additionally, or alternatively, a weight factor may be applied to the estimated patient data so that the estimated patient data may have a lower weight than corresponding measured patient data.

In some implementations, the unassigned patient data management system 100 may be utilized as an element of an Integrated Clinical Environment (ICE). As used herein, the “Integrated Clinical Environment (ICE)” refers to a platform to create a medical Internet of Things (IoT) associated with the care of a patient. In such an implementation, the unassigned patient data management system 100 may support many real-time clinical decision support algorithms. Additionally, or alternatively, the unassigned patient data management system 100 may support closed loop control algorithms of medical devices in the ICE.

In operation, the unassigned patient data match application 108 operates to collect data from a plurality of data sources in a plurality of formats. The unassigned patient data match application 108 converts data time stamps from the collected data to universal time zone data time stamps. The unassigned patient data match application 108 matches a same patient to the collected data based on the universal time zone data time stamps. The unassigned patient data match application 108 quantifies a quality estimate of the match and to transfers the match and quality estimate of the match to the user interface 122.

Advantageously, the unassigned patient data match application 108 seamlessly links data, regardless of structure and format, with relevant patients and patient EMRs.

In one implementation, data is aggregated, standardized, and then processed by a temporal matching algorithm to generate candidate patient confidence values. The candidate patients can be linked to the respective data automatically (e.g., based on confidence interval thresholds, etc.) or manually (e.g., on-the-fly validation by clinician, etc.).

In another implementation, a data aggregator receives data from all of the producers in a care facility network. In such an implementation, the received data is standardized and structured to prepare it for processing. This may include complex natural language processing (NLP), simple Regular Expression (Regex), and/or various other data pipeline techniques. For example, all standardized data is given a time-stamp label, regardless of final output structure.

In a further implementation, a temporal matching algorithm uses features of the standardized data along with the time-stamp label to predict a patient label. The temporal matching algorithm can include either or both rules-based and machine learning (ML) techniques. In such an implementation, the patient predictions are typically associated with a confidence interval, which may be used to integrate automations (e.g., based on thresholding, etc.) or provide further contextual information to reviewing users viewing outputs and/or associated data through a GUI.

As will be described in greater detail below, the unassigned patient data management system 100 implementation of the unassigned patient data match application 108 may include several operable components.

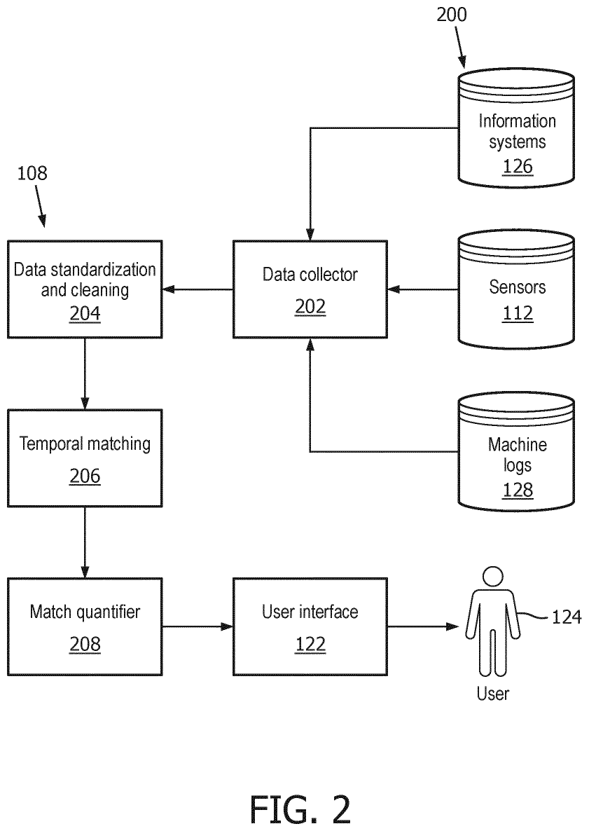

FIG. 2 is an illustration of a block diagram of another example unassigned patient data management system 200 according to an embodiment. As illustrated, the unassigned patient data management system 200 includes sensors 112, information system 126, machine log 128, and user interface 122. In some examples, the unassigned patient data management system 200 has several additional component including: a data collector 202, a data cleaner 204, a temporal match module 206, and a match quantifier 208.

In the illustrated implementation, the data collector 202 is to collect data from a plurality of data sources in a plurality of formats. For example, the operation to collect data from the plurality of data sources includes subscribing to a plurality of feeds associated with the plurality of data sources.

In one example, the data collector 202 is communicatively coupled to one or more of data sources. As discussed above, the data collector 202 may be communicatively coupled to the information system 126 (e.g., a Health Information System), the sensor 112 (e.g., a medical imaging device, a facility monitor system, etc.), machine log 128 (e.g., logs associated with a medical imaging system), a care platform, the like, and/or combinations thereof, for example. For example, the facility monitor system may include de-identified human presence data. Such de-identified human presence data includes a video-type camera feed, an infrared-type camera feed, a still-image type camera feed, an audio feed, the like, and/or combinations thereof.

For example, the data collector 202 is to collect data from various available sources. The data collector 202 collects data from many (or all) of the available sources in various formats by subscribing to the feeds of the data sources. For example, the data collector 202 may listen to HL7 and FHIR messages from a Hospital Information System (HIS), Digital Imaging and Communications in Medicine (DICOM) data (e.g., standardized format medical imaging information to transmit, store, retrieve, print, process, and display medical imaging information from a Picture Archiving and Communication System (PACS)/Radiology Information System (RIS)), de-identified audio/video feed (e.g., where the audio/video feed has been processed to prevent personal identity from being revealed to preserve privacy) from first and third party cameras/sensors, textual reports from machine logs, connection to the databases of various care platforms through SDKs/APIs, etc. Additionally, or alternatively, in situations where the care platform (e.g., Performance Management Data Platform, Virtualized Imaging Solution, Centralized Care Management System, EMR, etc.) is already connected to some of these data sources, the data collector 202 can tap in to this data via the care platform and complement it with data from other sources that the care platform is not connected to.

In some examples, the data cleaner 204 is communicatively coupled to the data collector 202. In such examples, the data cleaner 204 is to convert data time stamps from the collected data to universal time zone data time stamps. For example, the data cleaner 204 is further to parse information from the collected data, detect data outliers and/or data errors from the collected data, remove and/or fix detected data outliers and/or data errors from the collected data, and/or convert the collected data to a single format.

For example, the data cleaner 204 is to clean and standardize the data across sources. Since the data from the sources from the data collector 202 are typically in various formats, the data cleaner 204 will clean them up using rules-based methods to pre- and post-process the data and using statistical outlier detection algorithms to detect errors and standardize them to a simplified format such as JSON/XIL for ease of use.

In the illustrated implementation, the temporal match module 206 is communicatively coupled to the data cleaner 204. In such an implementation, the temporal match module 206 is to match a same patient to the collected data based on the universal time zone data time stamps. For example, the temporal match module is further to extract the universal time zone data time stamps, create a timeline of events based on the universal time zone data time stamps that were extracted, cluster data on a patient-by-patient basis based on the timeline of events, and/or mark the collected data with a patient identifier based on the clustered data. For example, the match quantifier is further to determine a probabilistic estimate and confidence interval of the match, receive user feedback regarding a reliability of the quality estimate of the match, remove the probabilistic estimate when below a probability threshold value, and/or create a complete timeline of care that associates the collected data from the plurality of data sources in the plurality of formats to the same patient.

For example, the temporal match module 206 is to match different data belonging to the same patient. Typically, the temporal match module 206 will employ one or more temporal matching algorithms to cluster the data from various sources to be identified as belonging to the same patient. Typically, all of the data sources mentioned above use timestamps so the temporal match module 206 can create a timeline of events for an individual patient in the care facility using these timestamps. In some examples, a care team can jump-start the training of the temporal match module 206 by manually time tracking various paths through the care facility and inputting sample data if such data is not available already through the various information systems.

In some examples, the match quantifier 208 is communicatively coupled to the temporal match module 206 and communicatively coupled the user interface 122. In such examples, the match quantifier 208 is to quantify a quality estimate of the match.

For example, the match quantifier 208 is to quantify the quality of the match. Typically, the match quantifier 208 will provide an indication of how good a given data match is using probabilistic estimates and confidence intervals (e.g., an estimation that the model is 95% confident the technologist who was scheduled to work in a certain exam room was occupied elsewhere and another technologist had to step in, so the patient's exam was performed by a different technologist than originally planned). Advantageously, such operations will make it easier for operational managers/clinicians to gain confidence into the insights they are getting from the collected data (e.g., if the technologist has likely changed and the replacement tech was more junior to the original, that could explain the need for multiple takes for the same image). Reinforcement learning algorithms can be used for this purpose (e.g., an intelligent agent can be trained by starting with providing a random score to a given match and by receiving a positive or negative feedback from the user and improving over time).

Additionally, or alternatively, the match quantifier 208 or some other module/component is to transfer the match and quality estimate of the match to the user interface 122. For example, the match quantifier 208 or some other module/component is to provide the collected data to the user (e.g., via a user interface/APIs). In such an example, a user interface will allow operational managers/clinicians to interact with the system and request data. Additionally, or alternatively, APIs are utilized to allow other applications to query the data automatically.

Timestamps are typically the most common element across various data sources. Some implementations described herein are directed to the use of temporal matching algorithms to connect various pieces of partial information about the patient that comes from different sources to create a complete picture of the patient care.

In operation, the need for manually sifting through the various sources of data to find information about a patient by the operational managers/clinicians is eliminated and the process is automated. Advantageously, uncertainty around the data is defined the unassigned patient data management system 200 through probabilistic estimations to inform the operations managers/clinicians of the quality of the match to a particular patient (e.g., a report that the model is 95% confident that the patient scheduled to a certain exam room that has another appointment with a specialist in 30 minutes has left the exam room 10 minutes ago).

### Detailed Description of Temporal Matching:

Let P be the set of persons (e.g., patients, staff) or clinical cases to be matched with a set of observations O(t) made at time t. An observation can be any information about a clinical procedure step that can be associated with a person or case. For example, an observation can be the start of an examination detected by a camera, without a unique identification of the patient. Since in general, multiple processes are running simultaneously, a number of observations related to different processes can be made at the same time.

In one realization it is assumed that for each observation (e.g., o∈O(t)), there is one person/case (e.g., p∈P) assigned. With k observations and n persons/cases to be assigned, a total number of

$$m = \frac{n !}{( n - k ) !}$$

combinations of assignments are possible. Let aᵢ 1≤i≤m be such a combination of assignments that uniquely assigns one person/case to each observation. For each aᵢ, a confidence score must be determined.

In the more general case, depending on the type of observations/available data, multiple persons/cases can be assigned to one observation. The proposed approach allows taking this generalization into account, by reducing the problem to determining confidence scores of specific observation—persons/case pairs (out of possibly multiple persons/cases per observation).

In some examples, the confidence score is composed of individual confidences for the assignment of each individual person/case, which can depend on all prior knowledge about the processes, such as: a planned clinical procedure type, a planned clinical procedure duration, assignments at a previous time, a planned schedule (order), a feature value coherence, the like, and/or combinations thereof.

In an example of the planned clinical procedure type: patient A is scheduled for an MR examination. Two observations of unknown patients are made. One in an MR examination room and one in a CT examination room. In this case, assigning patient A to the observation in the MR room is given a higher confidence score than assigning patient A to the observation in the CT room.

In an example of the planned clinical procedure duration: a sensor detects the presence of a patient on the MR table for 4 hours. This observation should typically get a very small confidence value. This assignment can be based on expected process durations (e.g., probabilistic look-up-tables).

In an example of the assignments at a previous time: some assignments are physically impossible when considering previous assignments, for example when a person would not be able to walk from the position of the previous observation to the position of the current observation within the time between the observations. In this case, all aᵢ that include this physically impossible assignment would typically receive a confidence score of zero or a very small confidence score that depends on the confidence of the previous assignment.

In an example of the planned schedule (order): in a situation where two patients are scheduled for examinations at different times, an observation in an examination room at the earlier time is more likely to correspond to the patient scheduled earlier, typically leading to a higher confidence score of this assignment.

In an example of the feature value coherence: a patient has a previous recorded weight of 80 kg and during the follow-up examination (e.g., after one week) of 120 kg. Then the confidence score of assigning that patient to this examination should typically get a very low confidence score.

Additionally, or alternatively, the confidence score can further be determined or refined using AI, for example by learning from historical assignments that have been verified manually.

The most probable assignment is typically then chosen as the one with the highest confidence score.

In one embodiment, a confidence score threshold value is defined. If the highest confidence score found for a certain set of assignments of observations is below that threshold, the quality of the assignment is considered insufficient. In this case, the assignment can be ignored, and the system waits for the next set of observations, or a user can be notified to validate and correct the assignment.

In one embodiment, the complete picture of the patient care is used to support decisions in real-time. For example, if the operations manager knows the status of an exam for a certain patient, they can plan the subsequent appointments of the same patient within the same day better.

In another embodiment, the gaps in patient knowledge is filled retroactively and the clinicians can make better care plans if they fully understand what care steps patients went through in a previous exam (e.g., the patient who was scheduled for an imaging exam in a certain room had difficulty staying still after a certain amount of time so the next time this patient is due for an imaging exam, the referring physician can recommend a Compressed Sense imaging procedure so that the exam is completed faster (patient might have difficulty admitting they had a hard time or not even remember they had difficulty but a sensor might still record such issues)).

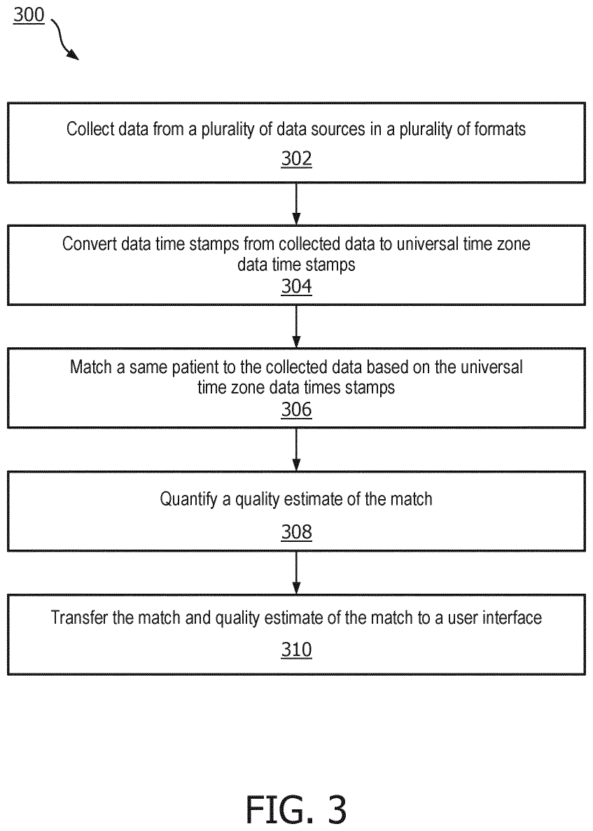

FIG. 3 shows an example method 300 for matching unassigned patient data to individual patients according to an embodiment. The method 300 may generally be implemented in the unassigned patient data management system 100 (FIG. 1) and/or the unassigned patient data management system 200 (FIG. 2), already discussed.

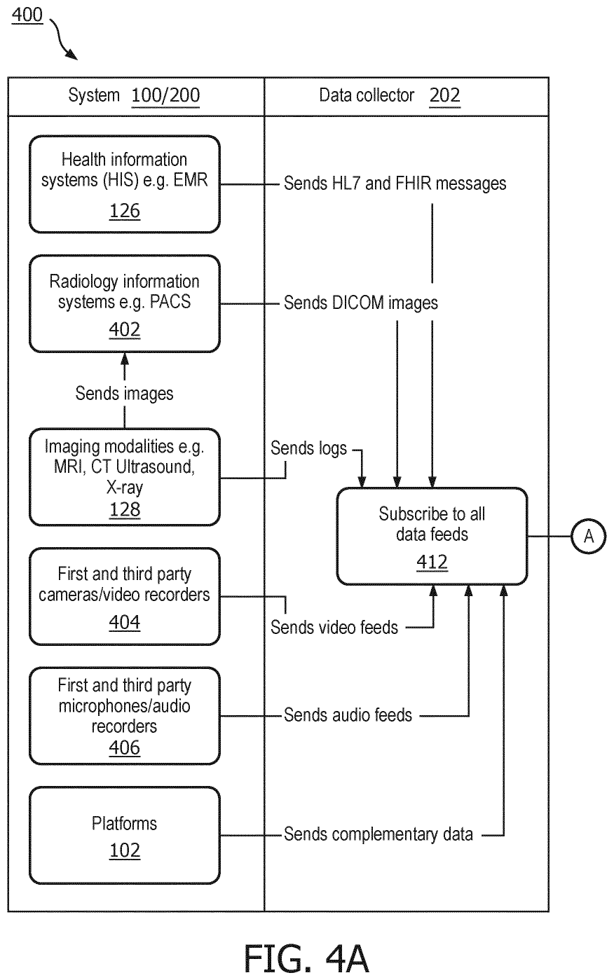

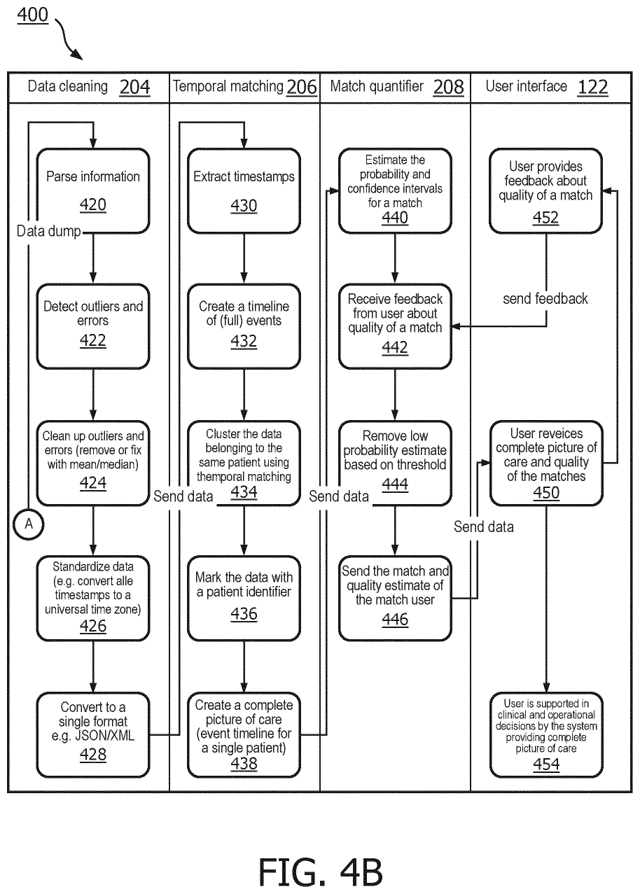

In an embodiment, the method 300 (as well as method 400 (FIGS. 4A and 4B), may be implemented in logic instructions (e.g., software), configurable logic, fixed-functionality hardware logic, etc., or any combination thereof.

Illustrated processing block 302 provides for collecting data from a plurality of data sources in a plurality of formats. For example, data may be collected from a plurality of data sources in a plurality of formats, via a data collector.

Illustrated processing block 304 provides for converting data time stamps from collected data to universal time zone data time stamps. For example, data time stamps from collected data may be converted to universal time zone data time stamps via a data cleaner communicatively coupled to the data collector.

Illustrated processing block 306 provides for matching a same patient to the collected data based on the universal time zone data time stamps. For example, a same patient may be matched to the collected data based on the universal time zone data time stamps, via a temporal match module communicatively coupled to the data cleaner.

Illustrated processing block 308 provides for quantifying a quality estimate of the match. For example, a quality estimate of the match may be quantified, via a match quantifier communicatively coupled to the temporal match module and a user interface.

Illustrated processing block 310 provides for transferring the match and quality estimate of the match to a user interface. For example, the match and quality estimate of the match may be transferred to the user interface, via the match quantifier.

In some examples, the methods described herein (e.g., method 300 and/or method 400) may be performed at least in part by cloud processing.

Additional and/or alternative operations for method 300 are described in greater detail below in the description of FIGS. 4A and 4B.

FIGS. 4A and 4B are a flowchart of an example of another method 400 for matching unassigned patient data to individual patients according to an embodiment. The method 400 may generally be implemented in the unassigned patient data management system 100 (FIG. 1) and/or the unassigned patient data management system 200 (FIG. 2), already discussed.

In an embodiment, the method 400 (as well as method 300 (FIG. 3) may be implemented in logic instructions (e.g., software), configurable logic, fixed-functionality hardware logic, etc., or any combination thereof. While certain portions of an unassigned patient data management system are illustrated in method 400, other portions of the unassigned patient data management system 100 (FIG. 1) and/or the unassigned patient data management system 200 (FIG. 2) have been intentionally left out to simplify the explanation of the method.

Illustrated processing block 412 provides for subscribing to a plurality of feeds associated with the plurality of data sources. For example, an operation by the data collector 202 to collect data from the plurality of data sources may include subscribing to a plurality of feeds associated with the plurality of data sources. For example, data may be collected from a plurality of data sources in a plurality of formats, via the data collector 202.

As discussed above, the data collector 202 may be communicatively coupled to the information system 126 (e.g., a Health Information System), one or more sensors (e.g., a medical imaging device, a facility monitor system, etc.), machine log 128 (e.g., logs associated with a medical imaging system), the care platform 102, the like, and/or combinations thereof, for example. For example, the facility monitor system may include de-identified human presence data. Such de-identified human presence data includes first and third party cameras/video recordings 404, first and third party microphones/audio recordings 406, the like, and/or combinations thereof.

For example, the data collector 202 collects data from many (or all) of the available sources in various formats by subscribing to the feeds of the data sources. For example, the data collector 202 may listen to HL7 and FHIR messages from the information system 126, Digital Imaging and Communications in Medicine (DICOM) data from a Radiology Information System (RIS)/Picture Archiving and Communication System (PACS) 402, de-identified audio/video feeds from the first and third party cameras/video recordings 404 and/or the first and third party microphones/audio recordings 406, textual reports from the machine logs 128, connection to the databases of the care platforms 102. Additionally, or alternatively, in situations where the care platform (e.g., Performance Management Data Platform, Virtualized Imaging Solution, Centralized Care Management System, EMR, etc.) is already connected to some of these data sources, the data collector 202 can tap in to this data via the care platforms 102 and complement it with data from other sources that the care platforms 102 are not connected to.

Illustrated processing block 420 provides for parsing information from the collected data. For example, information from the collected data may be parsed via the data cleaner 204 communicatively coupled to the data collector 202.

Illustrated processing block 422 provides for detecting data outliers and/or data errors from the collected data. For example, data outliers and/or data errors from the collected data may be detected via the data cleaner 204.

Illustrated processing block 424 provides for removing and/or fixing detected data outliers and/or data errors from the collected data. For example, detected data outliers and/or data errors from the collected data may be removed and/or fixed via the data cleaner 204.

Illustrated processing block 426 provides for converting data time stamps from collected data to universal time zone data time stamps. For example, data time stamps from collected data may be converted to universal time zone data time stamps via the data cleaner 204.

Illustrated processing block 428 provides for converting the collected data to a single format. For example, the collected data may be converted to a single format via the data cleaner 204.

Illustrated processing block 430 provides for extracting the universal time zone data time stamps. For example, the universal time zone data time stamps may be extracted via the temporal match module 206.

Illustrated processing block 432 provides for creating a timeline of events based on the universal time zone data time stamps that were extracted. For example, a timeline of events may be created based on the universal time zone data time stamps that were extracted via the temporal match module 206.

Illustrated processing block 434 provides for matching a same patient to the collected data based on the universal time zone data time stamps. For example, a same patient may be matched to the collected data based on the universal time zone data time stamps, via the temporal match module 206 communicatively coupled to the data cleaner 204. Additionally, or alternatively, processing block 434 provides for clustering data on a patient-by-patient basis based on the timeline of events.

Illustrated processing block 436 provides for marking the collected data with a patient identifier based on the clustered data. For example, the collected data may be marked with a patient identifier based on the clustered data via the temporal match module 206.

Illustrated processing block 438 provides for creating a complete timeline of care that associates the collected data from the plurality of data sources in the plurality of formats to the same patient. For example, a complete timeline of care may be created that associates the collected data from the plurality of data sources in the plurality of formats to the same patient via the temporal match module 206.

Illustrated processing block 440 provides for quantifying a quality estimate of the match. For example, a quality estimate of the match may be quantified, via the match quantifier 208 communicatively coupled to the temporal match module 206 and communicatively coupled to the user interface 122. Additionally, or alternatively, block 440 provides for determining a probabilistic estimate and confidence interval of the match.

Illustrated processing block 442 provides for receiving user feedback regarding a reliability of the quality estimate of the match. For example, user feedback may be received regarding a reliability of the quality estimate of the match from the user interface 122 via the match quantifier 208.

Illustrated processing block 444 provides for removing the probabilistic estimate when below a probability threshold value. For example, the probabilistic estimate may be removed when it is below a probability threshold value via the match quantifier 208.

Illustrated processing block 446 provides for transferring the match and quality estimate of the match to the user interface 122. For example, the match and quality estimate of the match may be transferred to the user interface 122, via the match quantifier 208.

Illustrated processing block 450 provides for receiving a completed picture of care and the quality of the matches for the same patient. For example, the completed picture of care and the quality of the matches for the same patient may be received via the user interface 122.

Illustrated processing block 452 provides for generating user feedback regarding a reliability of the quality estimate of the match. For example, user feedback may be generated regarding a reliability of the quality estimate of the match via the user interface 122.

Illustrated processing block 454 provides for supporting clinical and operational decisions in response to the provided complete picture of care. For example, clinical and operational decisions may be provided in response to the provided complete picture of care via the user interface 122.

Additionally, or alternatively, either as a separate module or as a part of the match quantifier 208, the unassigned patient data management system 100/200 further includes a decision support module. In one example, the decision support module is to transfer a notification that one or more medical imaging procedures are needed to the user interface and/or order an automated administration of the medical imaging procedures via one or more medical imaging devices in response to the complete timeline of care. In another example, the decision support module is to transfer a notification that one or more clinical interventions is needed to the user interface, order an automated administration of at least one of the one or more clinical interventions via one or more therapeutic devices, and/or order an automated administration of at least one of the one or more clinical interventions via one or more medical management devices in response to the complete timeline of care. In a further example, the decision support module is to transfer a notification to increase patient monitoring frequency to the user interface 122 and/or order an automated increase in patient monitoring frequency in response to the complete timeline of care. In such an example, the operation to increase the patient monitoring frequency comprises an increased data collection frequency and/or an increased data transmission frequency.

Additionally, or alternatively, method 400 provides for performing one or more interventions (not illustrated). In one example, one or more interventions may be performed via the therapeutic device 116 in response to a notification that the one or more clinical interventions are needed.

In another example, one or more interventions may be performed via the medical management device 118 in response a notification that the one or more clinical interventions are needed. For example, the medical management device 118 may supply and/or monitor the administration of one or more patient medications (e.g., blood pressure medications, diuretic medications, anti-anemia medications, cholesterol lowering medications, vitamin supplements, and/or the like).

In such an example, method 400 may order an automated administration of at least one of the one or more clinical interventions via one or more of the therapeutic devices 118, and/or order an automated administration of at least one of the one or more clinical interventions via one or more of the medical management devices 116.

In some implementations, a first threshold may be associated with a first clinical intervention and a second threshold may be associated with a second clinical intervention. For example, the first threshold provides a different intervention than the second threshold. Accordingly, individual clinical intervention are capable of being considered to take into account variations in the ‘costs’ (e.g., monetary cost, adverse health risks, and/or the like) and benefits (e.g., improved health potential) on an intervention-by intervention basis. The thresholds can be adjusted for each condition depending on the prevalence of the condition and the ‘cost’ of the action. For example, the ‘cost’ refers to a balance between benefits derived from the actions and the risks from the actions. Some actions are low risk (such as more frequent lab measurements) while some other actions (such as intubation) are associated with high risk. Therefore, the thresholds which trigger various actions can be dependent on the action associated with it on an action-by-action basis. Such procedures provide a framework to link machine learning based algorithm predictions to actions (e.g., interventions).

Additionally, or alternatively, the procedures described herein may provide a framework for clinical deployment of decision support algorithms. These procedures can work together with many clinical decision support (CDS) algorithms (such as AKI, ARDS, ADHF, HSI etc.). Clinical decision support (CDS) refers to computer-based support of clinical staff responsible for making decisions for the care of patients. Computer-based support for clinical decision-making staff may take many forms, from patient-specific visual/numeric health status indicators to patient-specific health status predictions and patient-specific health care recommendations. Further, the procedures described herein may be deployed on analytics platforms (such as Inference Engine, Critical Care Information System, Interoperability Solution, etc.) in conjunction with CDS algorithms.

It will be appreciated that some or all of the operations in method 500 above that have been described using a “pull” architecture (e.g., polling for new information followed by a corresponding response) may instead be implemented using a “push” architecture (e.g., sending such information when there is new information to report), and vice versa.

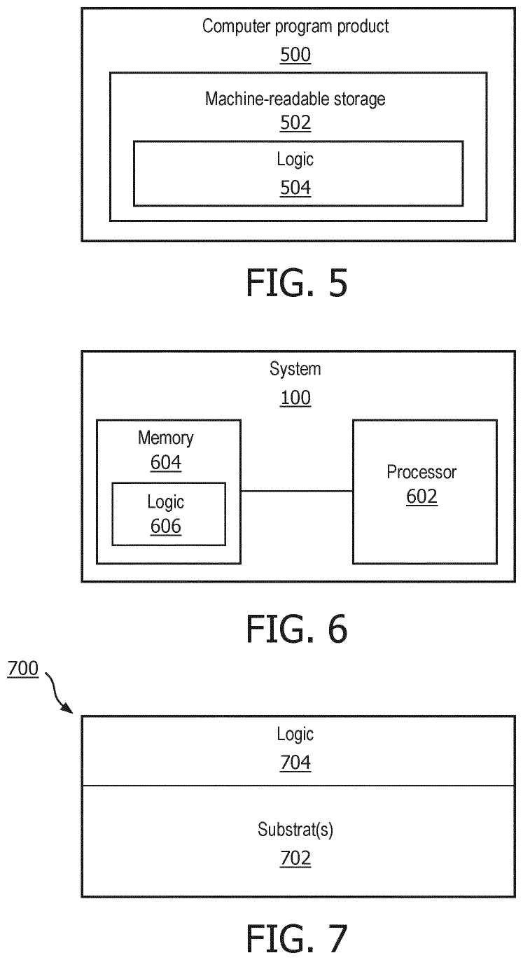

FIG. 5 illustrates a block diagram of an example computer program product 500. In some examples, as shown in FIG. 5, computer program product 500 includes a machine-readable storage 502 that may also include logic 504. In some implementations, the machine-readable storage 502 may be implemented as a non-transitory machine-readable storage. In some implementations the logic 504 may be implemented as machine-readable instructions, such as software, for example. In an embodiment, the logic 504, when executed, implements one or more aspects of the method 300 (FIG. 3), the method 400 (FIGS. 4A and 4B), and/or realize the system 100 (FIG. 1 and/or FIG. 2), already discussed.

FIG. 6 shows an illustrative example of the AKI management system 100. In the illustrated example, the AKI management system 100 may include a processor 602 and a memory 604 communicatively coupled to the processor 602. The memory 604 may include logic 606 as a set of instructions. In some implementations the logic 606 may be implemented as software. In an embodiment, the logic 606, when executed by the processor 602, implements one or more aspects of the method 300 (FIG. 3), the method 400 (FIGS. 4A and 4B), and/or realize the system 100 (FIG. 1 and/or FIG. 2), already discussed.

In some implementations, the processor 602 may include a general purpose controller, a special purpose controller, a storage controller, a storage manager, a memory controller, a micro-controller, a general purpose processor, a special purpose processor, a central processor unit (CPU), the like, and/or combinations thereof.

Further, implementations may include distributed processing, component/object distributed processing, parallel processing, the like, and/or combinations thereof. For example, virtual computer system processing may implement one or more of the methods or functionalities as described herein, and the processor 602 described herein may be used to support such virtual processing.

In some examples, the memory 604 is an example of a computer-readable storage medium. For example, memory 604 may be any memory which is accessible to the processor 602, including, but not limited to RAM memory, registers, and register files, the like, and/or combinations thereof. References to “computer memory” or “memory” should be interpreted as possibly being multiple memories. The memory may for instance be multiple memories within the same computer system. The memory may also be multiple memories distributed amongst multiple computer systems or computing devices.

FIG. 7 shows an illustrative semiconductor apparatus 700 (e.g., chip and/or package). The illustrated apparatus 700 includes one or more substrates 702 (e.g., silicon, sapphire, or gallium arsenide) and logic 704 (e.g., configurable logic and/or fixed-functionality hardware logic) coupled to the substrate(s) 702. In an embodiment, the logic 704 implements one or more aspects of the method 300 (FIG. 3), the method 400 (FIGS. 4A and 4B), and/or realize the system 100 (FIG. 1 and/or FIG. 2), already discussed.

In some implementations, logic 704 may include transistor array and/or other integrated circuit/IC components. For example, configurable logic and/or fixed-functionality hardware logic implementations of the logic 704 may include configurable logic such as, for example, programmable logic arrays (PLAs), field programmable gate arrays (FPGAs), complex programmable logic devices (CPLDs), or fixed-functionality logic hardware using circuit technology such as, for example, application specific integrated circuit (ASIC), complementary metal oxide semiconductor (CMOS) or transistor-transistor logic (TTL) technology, the like, and/or combinations thereof.

### Additional Notes and Examples

Example 1: An apparatus, comprising: a data collector to collect data from a plurality of data sources in a plurality of formats; a data cleaner communicatively coupled to the data collector, the data cleaner to convert data time stamps from the collected data to universal time zone data time stamps; a temporal match module communicatively coupled to the data cleaner, the temporal match module to match a same patient to the collected data based on the universal time zone data time stamps; and a match quantifier communicatively coupled to the temporal match module and a user interface, the match quantifier to quantify a quality estimate of the match, and to transfer the match and quality estimate of the match to the user interface.

Example 2: The apparatus of Example 1, wherein operations to collect data from the plurality of data sources comprises subscribing to a plurality of feeds associated with the plurality of data sources.

Example 3: The apparatus of any one of Examples 1 to 2, wherein the data collector is communicatively coupled to one or more of the following plurality of data sources: a Health Information System, a medical imaging device, a medical imaging system, a facility monitor system, and a care platform, and wherein the facility monitor system includes de-identified human presence data, wherein the de-identified human presence data includes one or more of the following: a video-type camera feed, an infrared-type camera feed, a still-image type camera feed, and an audio feed.

Example 4: The apparatus of any one of Examples 1 to 3, wherein the data cleaner is further to: parse information from the collected data, detect data outliers and/or data errors from the collected data, remove and/or fix detected data outliers and/or data errors from the collected data, and convert the collected data to a single format.

Example 5: The apparatus of any one of Examples 1 to 4, wherein the temporal match module is further to: extract the universal time zone data time stamps, create a timeline of events based on the universal time zone data time stamps that were extracted, cluster data on a patient-by-patient basis based on the timeline of events, and mark the collected data with a patient identifier based on the clustered data.

Example 6: The apparatus of any one of Examples 1 to 5, wherein the temporal match module is further to: create a complete timeline of care that associates the collected data from the plurality of data sources in the plurality of formats to the same patient.

Example 7: The apparatus of any one of Examples 1 to 6, wherein the match quantifier is further to: determine a probabilistic estimate and confidence interval of the match, receive user feedback regarding a reliability of the quality estimate of the match, and remove the probabilistic estimate when below a probability threshold value.

Example 8: The apparatus of any one of Examples 6 to 7, further comprising a decision support module to: transfer a notification to increase patient monitoring frequency to the user interface and/or order an automated increase in patient monitoring frequency in response to the complete timeline of care, wherein operations to increase the patient monitoring frequency comprises an increased data collection frequency and/or an increased data transmission frequency.

Example 9: The apparatus of any one of Examples 6 to 7, further comprising a decision support module to: transfer a notification that one or more medical imaging procedures are needed to the user interface and/or order an automated administration of the medical imaging procedures via one or more medical imaging devices in response to the complete timeline of care.

Example 10: The apparatus of any one of Examples 6 to 7, further comprising a decision support module to: transfer a notification that one or more clinical interventions is needed to the user interface, order an automated administration of at least one of the one or more clinical interventions via one or more therapeutic devices, and/or order an automated administration of at least one of the one or more clinical interventions via one or more medical management devices in response to the complete timeline of care.

Example 11: A method, comprising: collecting, via a data collector, data from a plurality of data sources in a plurality of formats; converting, via a data cleaner communicatively coupled to the data collector, data time stamps from collected data to universal time zone data time stamps; matching, via a temporal match module communicatively coupled to the data cleaner, a same patient to the collected data based on the universal time zone data time stamps; quantifying, via a match quantifier communicatively coupled to the temporal match module and a user interface, a quality estimate of the match; and transferring, via the match quantifier, the match and quality estimate of the match to the user interface.

Example 12: The method of Example 11, wherein operations to collect data from the plurality of data sources comprises subscribing to a plurality of feeds associated with the plurality of data sources, wherein the plurality of data sources includes one or more of: a Health Information System, a medical imaging device, a medical imaging system, a facility monitor system, and a care platform, and wherein the facility monitor system includes de-identified human presence data, wherein the de-identified human presence data includes one or more of the following: a video-type camera feed, an infrared-type camera feed, a still-image type camera feed, and an audio feed.

Example 13: The method of any one of Examples 11 to 12, further comprising: parsing information from the collected data, detecting data outliers and/or data errors from the collected data, removing and/or fixing detected data outliers and/or data errors from the collected data, and converting the collected data to a single format.

Example 14: The method of any one of Examples 11 to 13, further comprising: extracting the universal time zone data time stamps, creating a timeline of events based on the universal time zone data time stamps that were extracted, clustering data on a patient-by-patient basis based on the timeline of events, marking the collected data with a patient identifier based on the clustered data, and creating a complete timeline of care that associates the collected data from the plurality of data sources in the plurality of formats to the same patient.

Example 15: The method of any one of Examples 11 to 14, further comprising: determining a probabilistic estimate and confidence interval of the match, receiving user feedback regarding a reliability of the quality estimate of the match, and removing the probabilistic estimate when below a probability threshold value.

Example 16: At least one computer readable medium, comprising a set of instructions, which when executed by a computing device, cause the computing device to: collect data from a plurality of data sources in a plurality of formats; convert data time stamps from collected data to universal time zone data time stamps; match a same patient to the collected data based on the universal time zone data time stamps; quantify a quality estimate of the match; and transfer the match and quality estimate of the match to a user interface.

Example 17: The at least one computer readable medium of Example 16, wherein the set of instructions, which when executed by the computing device, cause the computing device further to: subscribe to a plurality of feeds associated with the plurality of data sources, wherein the plurality of data sources includes one or more of: a Health Information System, a medical imaging device, a medical imaging system, a facility monitor system, and a care platform, and wherein the facility monitor system includes de-identified human presence data, wherein the de-identified human presence data includes one or more of the following: a video-type camera feed, an infrared-type camera feed, a still-image type camera feed, and an audio feed.

Example 18: The at least one computer readable medium of any one of Examples 11 to 17, wherein the set of instructions, which when executed by the computing device, cause the computing device further to: parse information from the collected data, detect data outliers and/or data errors from the collected data, remove and/or fix detected data outliers and/or data errors from the collected data, and convert the collected data to a single format.

Example 19: The at least one computer readable medium of any one of Examples 11 to 18, wherein the set of instructions, which when executed by the computing device, cause the computing device further to: extract the universal time zone data time stamps, create a timeline of events based on the universal time zone data time stamps that were extracted, cluster data on a patient-by-patient basis based on the timeline of events, mark the collected data with a patient identifier based on the clustered data, and create a complete timeline of care that associates the collected data from the plurality of data sources in the plurality of formats to the same patient.

Example 20: The at least one computer readable medium of any one of Examples 11 to 19, wherein the set of instructions, which when executed by the computing device, cause the computing device further to: determine a probabilistic estimate and confidence interval of the match, receive user feedback regarding a reliability of the quality estimate of the match, and remove the probabilistic estimate when below a probability threshold value.

Example 21 includes an apparatus comprising means for performing the method of any one of Examples 11 to 15.

Example 22 includes a machine-readable storage including machine-readable instructions, which when executed, implement a method or realize an apparatus as claimed in any preceding claim.

All definitions, as defined and used herein, should be understood to control over dictionary definitions, definitions in documents incorporated by reference, and/or ordinary meanings of the defined terms.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. The term “coupled” may be used herein to refer to any type of relationship, direct or indirect, between the components in question, and may apply to electrical, mechanical, fluid, optical, electromagnetic, electromechanical, or other connections. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components.

In the claims, as well as in the specification above, the terms “first”, “second”, etc. may be used herein only to facilitate discussion, and carry no particular temporal or chronological significance unless otherwise indicated.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The indefinite articles “a” and “an,” as used herein in the specification and in the claims, unless clearly indicated to the contrary, should be understood to mean “at least one.”

As used herein, the term “or” or “and/or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used in this application and in the claims, a list of items joined by the term “one or more of” may mean any combination of the listed terms. For example, the phrases “one or more of A, B or C” may mean A; B; C; A and B; A and C; B and C; or A, B and C.

As is described above in greater detail, one or more processor, other unit, the like, and/or combinations thereof may fulfill the functions of several items recited in the claims.

As is described above in greater detail, a computer program may be stored/distributed on a suitable computer readable medium, such as an optical storage medium or a solid-state medium supplied together with or as part of other hardware, but may also be distributed in other forms, such as via the Internet or other wired or wireless telecommunication systems.

It should also be understood that, unless clearly indicated to the contrary, in any methods discussed herein that include more than one step or act, the order of the steps or acts of the method is not necessarily limited to the order in which the steps or acts of the method are recited. Further, such methods may include additional or alternative steps or acts.

As used in the claims, the mere fact that certain measures are recited in mutually different dependent claims does not indicate that a combination of these measured cannot be used to advantage.

It is also noted that the claims may include reference signs/numerals in accordance with PCT Rule 6.2(b). However, the present claims should not be considered to be limited to the exemplary embodiments corresponding to the reference signs/numerals.

Those skilled in the art will appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. An apparatus, comprising:
a data collector to collect data from a plurality of data sources in a plurality of formats;
a data cleaner communicatively coupled to the data collector, the data cleaner to convert data time stamps from the collected data to universal time zone data time stamps;
a temporal match module communicatively coupled to the data cleaner, the temporal match module to match a same patient to the collected data based on the universal time zone data time stamps; and
a match quantifier communicatively coupled to the temporal match module and a user interface, the match quantifier to quantify a quality estimate of the match, and to transfer the match and quality estimate of the match to the user interface.

2. The apparatus of claim 1, wherein operations to collect data from the plurality of data sources comprises subscribing to a plurality of feeds associated with the plurality of data sources.

3. The apparatus of claim 1, wherein the data collector is communicatively coupled to one or more of the following plurality of data sources: a Health Information System, a medical imaging device, a medical imaging system, a facility monitor system, and a care platform, and
wherein the facility monitor system includes de-identified human presence data, wherein the de-identified human presence data includes one or more of the following: a video-type camera feed, an infrared-type camera feed, a still-image type camera feed, and an audio feed.

4. The apparatus of claim 1, wherein the data cleaner is further to:
parse information from the collected data,
detect data outliers and/or data errors from the collected data,
remove and/or fix detected data outliers and/or data errors from the collected data, and
convert the collected data to a single format.

5. The apparatus of claim 1, wherein the temporal match module is further to:
extract the universal time zone data time stamps,
create a timeline of events based on the universal time zone data time stamps that were extracted,
cluster data on a patient-by-patient basis based on the timeline of events, and
mark the collected data with a patient identifier based on the clustered data.

6. The apparatus of claim 1, wherein the temporal match module is further to:
create a complete timeline of care that associates the collected data from the plurality of data sources in the plurality of formats to the same patient.

7. The apparatus of claim 1, wherein the match quantifier is further to:
determine a probabilistic estimate and confidence interval of the match,
receive user feedback regarding a reliability of the quality estimate of the match, and
remove the probabilistic estimate when below a probability threshold value.

8. The apparatus of claim 6, further comprising a decision support module to: transfer a notification to increase patient monitoring frequency to the user interface and/or order an automated increase in patient monitoring frequency in response to the complete timeline of care, wherein operations to increase the patient monitoring frequency comprises an increased data collection frequency and/or an increased data transmission frequency.

9. The apparatus of claim 6, further comprising a decision support module to: transfer a notification that one or more medical imaging procedures are needed to the user interface and/or order an automated administration of the medical imaging procedures via one or more medical imaging devices in response to the complete timeline of care.

10. The apparatus of claim 6, further comprising a decision support module to: transfer a notification that one or more clinical interventions is needed to the user interface, order an automated administration of at least one of the one or more clinical interventions via one or more therapeutic devices, and/or order an automated administration of at least one of the one or more clinical interventions via one or more medical management devices in response to the complete timeline of care.

11. A method, comprising:
collecting, via a data collector, data from a plurality of data sources in a plurality of formats;
converting, via a data cleaner communicatively coupled to the data collector, data time stamps from collected data to universal time zone data time stamps;
matching, via a temporal match module communicatively coupled to the data cleaner, a same patient to the collected data based on the universal time zone data time stamps;
quantifying, via a match quantifier communicatively coupled to the temporal match module and a user interface, a quality estimate of the match; and
transferring, via the match quantifier, the match and quality estimate of the match to the user interface.

12. The method of claim 11, wherein operations to collect data from the plurality of data sources comprises subscribing to a plurality of feeds associated with the plurality of data sources,
wherein the plurality of data sources includes one or more of: a Health Information System, a medical imaging device, a medical imaging system, a facility monitor system, and a care platform, and
wherein the facility monitor system includes de-identified human presence data, wherein the de-identified human presence data includes one or more of the following: a video-type camera feed, an infrared-type camera feed, a still-image type camera feed, and an audio feed.

13. The method of claim 11, further comprising:
parsing information from the collected data,
detecting data outliers and/or data errors from the collected data,
removing and/or fixing detected data outliers and/or data errors from the collected data, and
converting the collected data to a single format.

14. The method of claim 11, further comprising:
extracting the universal time zone data time stamps,
creating a timeline of events based on the universal time zone data time stamps that were extracted,
clustering data on a patient-by-patient basis based on the timeline of events,
marking the collected data with a patient identifier based on the clustered data, and
creating a complete timeline of care that associates the collected data from the plurality of data sources in the plurality of formats to the same patient.

15. The method of claim 11, further comprising:
determining a probabilistic estimate and confidence interval of the match,
receiving user feedback regarding a reliability of the quality estimate of the match, and
removing the probabilistic estimate when below a probability threshold value.

16. At least one computer readable medium, comprising a set of instructions, which when executed by a computing device, cause the computing device to:
collect data from a plurality of data sources in a plurality of formats;
convert data time stamps from collected data to universal time zone data time stamps;
match a same patient to the collected data based on the universal time zone data time stamps;
quantify a quality estimate of the match; and
transfer the match and quality estimate of the match to a user interface.

17. The at least one computer readable medium of claim 16, wherein the set of instructions, which when executed by the computing device, cause the computing device further to:
subscribe to a plurality of feeds associated with the plurality of data sources,
wherein the plurality of data sources includes one or more of: a Health Information System, a medical imaging device, a medical imaging system, a facility monitor system, and a care platform, and
wherein the facility monitor system includes de-identified human presence data, wherein the de-identified human presence data includes one or more of the following: a video-type camera feed, an infrared-type camera feed, a still-image type camera feed, and an audio feed.

18. The at least one computer readable medium of claim 16, wherein the set of instructions, which when executed by the computing device, cause the computing device further to:
parse information from the collected data,
detect data outliers and/or data errors from the collected data,
remove and/or fix detected data outliers and/or data errors from the collected data, and
convert the collected data to a single format.

19. The at least one computer readable medium of claim 16, wherein the set of instructions, which when executed by the computing device, cause the computing device further to:
extract the universal time zone data time stamps,
create a timeline of events based on the universal time zone data time stamps that were extracted,
cluster data on a patient-by-patient basis based on the timeline of events,
mark the collected data with a patient identifier based on the clustered data, and
create a complete timeline of care that associates the collected data from the plurality of data sources in the plurality of formats to the same patient.

20. The at least one computer readable medium of claim 16, wherein the set of instructions, which when executed by the computing device, cause the computing device further to:
determine a probabilistic estimate and confidence interval of the match,
receive user feedback regarding a reliability of the quality estimate of the match, and
remove the probabilistic estimate when below a probability threshold value.
